SEQUENCE LISTING (4 sequences)

SEQ ID NO: 1 - protein, 20 aa, Artificial Sequence
  Peptide
VVYRRVRCRRKGGIRFAING

SEQ ID NO: 2 - protein, 24 aa, Artificial Sequence
  Peptide
ALLKVLFSWFRKHNSRRKNLPAKN

SEQ ID NO: 3 - protein, 20 aa, Artificial Sequence
  Peptide
GWFNRGARCGRMIKIKARNG

SEQ ID NO: 4 - protein, 20 aa, Artificial Sequence
  Peptide
AYRRVPCRKRGGIRFTINGH

(19) United States
(12) Patent Application Publication
(10) Pub. No.: US 2023/0287061 A1
(43) Pub. Date: Sep. 14, 2023

(54) NEW ANTIMICROBIAL PEPTIDES

(71) Applicant: Università Cattolica del Sacro Cuore, Milano (IT)

(72) Inventors: Daniela BASSI, Cremona (IT); Pier Sandro COCCONCELLI, Piacenza (IT); Francesca DILDA, Cremona (IT); Francesca BUGLI, Rome (IT); Luigi ORRU', Fiorenzuola d'Arda (IT); Antonella LAMONTANARA, Fiorenzuola d'Arda (IT)

(73) Assignee: Università Cattolica del Sacro Cuore, Milano (IT)

(21) Appl. No.: 18/041,357

(22) PCT Filed: Aug. 9, 2021
(86) PCT No.: PCT/IB2021/057317

§ 371 Date: Feb. 10, 2023

(30) Foreign Application Priority Data
Aug. 10, 2020 (IT) 102020000019921

(51) Int. Cl. C07K 14/415; A01N 63/50; A01P 1/00; A61P 31/04

(52) U.S. Cl. CPC C07K 14/415; A01N 63/50; A01P 1/00; A61P 31/04

(57) ABSTRACT

The present invention relates to new antimicrobial peptides of plant origin, with bacteriostatic and/or bactericidal effect against Gram positive and Gram negative bacteria, compositions including them and use thereof.

The present invention relates to new antimicrobial peptides of plant origin, having bacteriostatic and/or bactericidal effect against Gram positive and Gram negative bacteria, compositions including them and use thereof.

### STATE OF THE ART

In Europe, foodborne illnesses are one of the major health problems. By virtue of their chemical-physical characteristics, foods are subject to colonization by harmful microorganisms that exert their effects both as responsible for the processes of alteration and degradation of food, and as pathogens capable of causing food poisoning. These manifest themselves with a diversity of clinical pictures ranging from mild symptoms to lethality. The most common food infections are those caused by Campylobacter, Listeria, Salmonella, Escherichia coli, Bacillus cereus, Clostridium botulinum. With over 246,000 cases reported each year in humans, Campylobacter is the most frequent foodborne disease in Europe, just as listerioses, for example, represent a constant health problem; in 2015 alone, 27 member states reported 2,726 confirmed cases of listeriosis in humans due to contaminated food, with a mortality of 18.8%. At the same time, microorganisms are responsible for degradation processes that alter the organoleptic qualities of foods, shortening their shelf-life and introducing possible resistance to antibiotics in the food chain. Contamination risk is present along the entire supply chain that goes from production to consumption and therefore, in addition to control interventions, also prevention interventions are necessary.

In recent years there has been a growing interest in bio-control systems that use peptides produced by bacteria, plants and insects as natural antimicrobials in food. These peptides are generally small molecules of 15-50 amino acids, showing interesting biological properties, such as, for example, bacteriostatic and/or bactericidal properties even on organisms that may show resistance to antibiotics. In particular, plant organisms are strong producers of said compounds, representing an excellent source from which to draw natural compounds to be used against unwanted microorganisms of food interest without altering the organoleptic characteristics of the food.

Furthermore, potential advantages of the latter, in addition to a different mechanism of action and a broader spectrum, are linked to their lower allergenicity and their use as alternatives to chemical preservatives and additives. Currently, the problem of microbial contamination of food is often solved by heat treatments to counter the development of pathogenic and alterative organisms in food. However, these treatments, in addition to involving a high energy expenditure by industries, with a consequent economic and environmental impact, are often the cause of an important loss of factors that characterize the quality of food products, altering their organoleptic and nutritional characteristics. In addition, there is a strong need for producers to reduce the use of synthetic compounds with antimicrobial activity and of chemical additives, driven both by the onset of more and more allergies to these substances, as by the need to produce foods that preserve the characteristics of authenticity and naturalness and that meet the current taste of consumers.

The search for innovative bio-control methods (techniques based on the use of microorganisms and/or their metabolites) to produce safe, genuine and preservable food represents an added value for the manufacturing companies and would constitute a further step forward in the fight against food waste of which microbial contamination is one of the main causes.

Furthermore, due to the extensive use of antibiotics starting from the food supply chains to medical treatments, the emergence of pathogenic organisms resistant to antibiotics is increasingly common in the medical field (human and veterinary), with serious consequences on public and animal health.

The identification of compounds with antibacterial/bactericidal activity of natural origin, as alternatives to antibiotics and/or chemical or thermal treatments of food products or of environments or of plants, is hence of great interest both for the food field and for the human and veterinary clinical field, in order to counter contamination and microbial infections and prevent the development of further antibiotic resistance.

### SUMMARY OF THE INVENTION

The authors of the present invention have identified chemical-physical parameters for selecting peptides of plant origin which show antimicrobial activity (bacteriostatic and/or bactericidal) towards Gram positive and Gram negative bacteria.

In fact, the authors have isolated peptides from edible plants that fall within the aforementioned parameters and that have shown bacteriostatic and/or bactericidal activity against numerous types of bacteria, both Gram positive and Gram negative.

Said peptides, being derived from edible plants, in particular from vegetables commonly used in human and animal nutrition, have the advantage of being intrinsically not harmful to humans and animals and of being suitable, for example, to be mixed with aliments in order to exert their antibacterial action, allowing not to alter the organoleptic and nutritional characteristics of the same. Said peptides therefore respond both to the need of increasing the shelf life of foods by slowing down their microbiological deterioration and to the need of counteracting the development of pathogenic bacteria that are harmful to health.

Furthermore, said peptides can be used, again by virtue of their broad-spectrum bacteriostatic and/or bactericidal activity, for the sanitization of environments in which bacterial contamination may exist, such as for example environments for animal breeding, environments and plants of the food industry, hospital environments and veterinary clinic environments.

Further, said peptides can be used for the treatment of water such as drinking water and water for animal breeding.

Finally, said peptides can be used in the medical, human and veterinary fields, for the treatment, or to assist in the treatment, against bacterial infections, alternatively or concomitantly with antibiotic treatments.

Therefore, object of the invention are:

A peptide derived from edible plants wherein said peptide has a length from 18 to 27 amino acids, and comprises from 30 to 40% positively charged amino acids, from 9 to 14% aromatic amino acids and 0% negatively charged amino acids;

a method for the identification of peptides of natural origin having bacteriostatic and/or bactericidal activity, including the following steps
a. analysing the sequence of peptides of plant origin, preferably derived from edible plants, and selecting among these, peptides whose sequence has the following features:
a length from 18 to 27 amino acids wherein, from 30 to 40% of said amino acid sequence consisting of positively charged amino acids, from 9 to 14% of said amino acid sequence consisting of aromatic amino acids and said amino acid sequence being free of negatively charged amino acids;
b. calculating the net charge, the isoelectric point, the hydrophobicity and the Boman index of each peptide selected in point a. and further selecting peptides with Boman index between 2 and 4, a net positive charge between 5.5 and 8, and a hydrophobicity between 0 and −1 and an isoelectric point between 11 and 13.
a bacteriostatic and/or bactericidal antibacterial composition comprising one or more of said peptides and at least one excipient and/or carrier;
a bacteriostatic and/or bactericidal antibacterial composition comprising one or more of said peptides and at least one pharmaceutically acceptable excipient and/or carrier for use as a medicament,
the use of a bacteriostatic and/or bactericidal antibacterial composition comprising one or more of said peptides and at least one excipient and/or carrier for the sanitization of environments or systems;
a method for the antibacterial treatment of foods comprising mixing said foods or treating said foods with one or more of said peptides or with said composition;
a method for sanitizing water comprising mixing said waters or treating said waters with one or more of said peptides or with said composition;
foodstuff comprising one or more of said peptides or said composition.

### GLOSSARY

The term edible plants according to the present invention indicates plants commonly used for human or animal nutrition, it can therefore be replaced, in any part of the description and claims, by the expression “plants for human and/or animal alimentary use”.

The term “derived from plants” such as edible plants, according to the present invention, means that the peptide object of the invention can be isolated from said plants or even reproduced recombinantly or by artificial synthesis once isolated and identified from the aforementioned plants. It is therefore not necessary that at each use the peptide is isolated from the plant of origin from which it was first isolated but it can also be produced according to standard genetic engineering techniques commonly used by the skilled person or by artificial synthesis.

Peptide 1 in the present description it is synonymous with peptide having SEQ ID NO 1

Peptide 2 in the present description it is synonymous with peptide having SEQ ID NO 2

Peptide 3 in the present description it is synonymous with peptide having SEQ ID NO 3

Peptide 4 in the present description it is synonymous with peptide having SEQ ID NO 4

According to the present invention, the term amino acids refers to the 20 natural proteinogenic amino acids, namely Glycine, Alanine, Valine, Leucine, Isoleucine, Methionine, Phenylalanine, Tyrosine, Tryptophan, Serine, Proline, Threonine, Cysteine, Asparagine, Glutamine, Lysine, Histidine , Arginine, Aspartic Acid and Glutamic Acid.

It is known that amino acids are divided into subgroups according to their properties, according to the present invention the amino acids are divided into non-polar or uncharged, aromatic, positively charged or negatively charged according to the definition provided in the IARC TP53 database of the WHO, and are hence divided as shown below.

According to the present invention, the non-polar/apolar or non-charged amino acids consist of the following amino acids:

| | |
|---|---|
| Glycine | .G |
| Alanine | A |
| Valine | V |
| Leucine | L |
| Isoleucine | I |
| Methionine | M |
| Serine | S |
| Proline | P |
| Threonine | T |
| Cyisteine | C |
| Asparagine | N |
| Glutamine | Q; |

aromatic amino acids consists of the following amino acids

| | |
|---|---|
| Phenylalanine | F |
| Tyrosine | Y |
| Triptophane | W; |

positively charged amino acids consists of the following amino acids:

| | |
|---|---|
| Lysine | K |
| Histidine | H |
| Argininae | R; e |

negatively charged amino acids consists of the following amino acids:

| | |
|---|---|
| Aspartic acid (aspartate) | D |
| Glutamic acid (glutamate) | E. |

Boman index according to the present description i sas defined by Hans Boman in H. G. Boman Journal of Internal Medicine 2003; 254: 197-215, REVIEW Antibacterial peptides: basic facts and emerging concepts. Calculation of the index is explained in Table 2 of the paper and a simple freely available program for calculation of the index has been developed by the author as disclosed in the paper.The index assesses a value of binding potential for peptides with antibacterial activity.

### DETAILED DESCRIPTION OF THE FIGURES

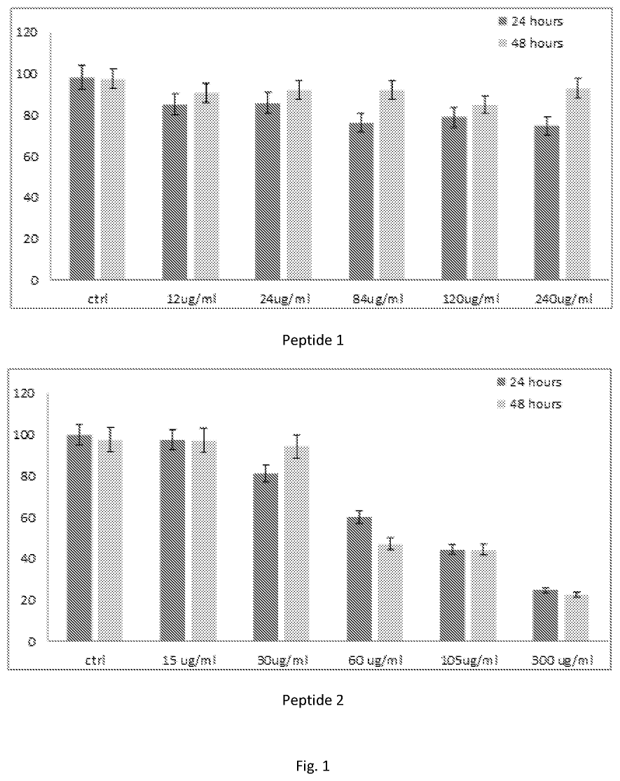

FIG. 1 shows the results of cytotoxicity studies of peptides having SEQ ID NO 1 and SEQ ID NO 2 on human 3T3 fibroblasts at different peptide concentrations. The numerical values indicate the cell viability percentage (in ordinate) at the different concentrations of peptide tested (expressed in μg/ml). Ctrl: control treated with the culture medium only. Data is shown as the mean±standard deviation.

### DETAILED DESCRIPTION OF THE SEQUENCES

SEQ ID NO 1
VVYRRVRCRRKGGIRFAING
SEQ ID NO 2
ALLKVLFSWFRKHNSRRKNLPAKN
SEQ ID NO 3
GWFNRGARCGRMIKIKARNG
SEQ ID NO 4
AYRRVPCRKRGGIRFTINGH

### DETAILED DESCRIPTION

The authors of the present invention have identified, as mentioned above, some genetic and chemical-physical parameters, which allow to select, among all the peptides produced by edible plants, for food use, peptides with antimicrobial ability, and in particular antibacterial, bacteriostatic and/or bactericidal.

The present invention, therefore relates, to peptides isolated from edible plants, having bacteriostatic and/or bactericidal activity, wherein said peptides have, each, the following features, i.e. they have a length from 18 to 27 amino acids, and from 30 to 40% of their amino acid sequence is composed of positively charged amino acids, 9 to 14% of their amino acid sequence is composed of aromatic amino acids and 0% of their amino acid sequence is composed of negatively charged amino acids.

According to the invention, in any embodiment reported below, the peptides of the invention preferably have a length from 19 to 25, or from 20 to 24 amino acids.

According to the present invention, aromatic amino acids mean the following amino acids

| | |
|---|---|
| Phenylalanine | F |
| Tyrosine | Y |
| Triptophane | W; |

In any part of the description the phrase . . . 0% of their amino acid sequence is composed of negatively charged amino acids, referred to the peptides of the invention, can be replaced by the expression “said peptides being free of negatively charged amino acids” or other expressions describing the absence of negatively charged amino acids from the peptide of the invention.

The amino acid class indicated in the present description (for example aromatics, positively charged etc., as reported in the glossary) can be replaced in any part of the description and claims with the group of specific amino acids indicated in the glossary for each class.

In one embodiment, said positively charged amino acids are from 30 to 35% of the amino acid sequence of each of said peptides, and said aromatic amino acids are from 10 to 12.5% of the amino acid sequence of each of said peptides.

According to the present invention, moreover, said peptides preferably have a Boman index between 2 and 4, a net positive charge between 5.5 and 8, a hydrophobicity between 0 and −1 and an isoelectric point between 11 and 13 .

According to an embodiment of the invention, for values in which the measured value may vary depending on the pH at which the measurement is carried out, the indicated values are understood to be calculated at pH7.

The isoelectric point and hydrophobicity of a peptide can be easily calculated from the peptide sequence itself with standard methods.

By hydrophobicity in this description and in the claims the average hydrophobicity is meant.

Programs for the calculation of said values area also available n the internet, such as, by way of example, https://www.bachem.com/knowledge-center/peptide-calculator and other similar sites or by using commercially available programs such as R Peptides, as described by Osorio et al 2015 (Osorio, D., Rondon-Villarreal, P. & Torres, R. Peptides: A package for data mining of antimicrobial peptides. The R Journal. 7(1), 4-14 (2015)). According to an embodiment of the invention, hydrophobicity is calculated using the Eisemberg hydrophobicity scale (Eisenberg D., Schwarz E., Komarony M., Wall R. Reference: J. Mol. Biol. 179:125-142(1984) which indicates precise values for each amino acid as reported below:
Ala: 0.620 Arg: −2.530 Asn: −0.780 Asp: −0.900 Cys: 0.290 Gln: −0.850 Glu: −0.740 Gly: 0.480 His: −0.400 Ile: 1.380 Leu: 1.060 Lys: −1.500 Met: 0.640 Phe: 1.190 Pro: 0.120 Ser: −0.180 Thr: −0.050 Trp: 0.810 Tyr: 0.260 Val: 1.080, therefore in an embodiment of the invention the hydrophobicity value is calculated using the Eisenberg scale.

Net charge and isoelectric point can be calculated using any of the nine pKa scales, in a preferred embodiment the EMBOSS scale known to the skilled in the art is used as described in Osorio et al 2015.

According to the present invention, the net charge is preferably calculated at pH 7.

Advantageously, each peptide of the present invention, as mentioned above, exhibits antibacterial, bacteriostatic and/or bactericidal activity.

According to the invention, the peptide is isolated from edible plants or from conventional systems that allow the expression of the aforementioned peptide or its synthesis (therefore the peptide, once isolated from the plant can be expressed recombinantly and isolated from the recombinant expression system).

The peptide is therefore understood as isolated from its plant of origin and the term “derived from edible plants” in the description indicates that the origin of the peptide is an edible plant without limiting its subsequent expression and isolation by recombinant or artificial systems.

In a preferred embodiment, said antibacterial activity (bacteriostatic and/or bactericidal) is detectable both towards Gram negative bacteria and towards Gram positive bacteria.

In a particular embodiment, the peptide of the invention is a peptide having SEQ ID NO 1, SEQ ID NO 2, SEQ ID NO 3 or SEQ ID NO 4 or variants thereof as herein defined.

In a preferred embodiment, said peptide is a peptide having SEQ ID NO 1 or SEQ ID NO 2 or variants thereof as defined herein.

Also object of the invention are variants of the peptides having SEQ ID NO 1-4, each of said variants being a peptide having 80%, 90%, 95%, 97%, 98%, 99% sequence identity with one of peptides having SEQ ID NO 1-4. These variants can have a length of from 18 to 27 amino acids, preferably from 18 to 25, even more preferably from 19 to 25, or from 20 to 24 amino acids.

The variants of the peptides having SEQ ID 1-4 according to the invention meet the requirements indicated for the peptides object of the invention, and therefore they preferably are peptides consisting of positively charged amino acids from 30 to 40%, preferably from 30 to 35% of the their amino acid sequence, from 9 to 14% aromatic amino acids, preferably from 10 to 12.5% of their amino acid sequence and 0% of their amino acid sequence is composed of negatively charged amino acids, i.e. they are free of negatively charged amino acids.

Furthermore, according to the present invention, said variants are preferably peptides with a Boman index between 2 and 4, a net positive charge between 5.5 and 8, a hydrophobicity between 0 and −1 and an isoelectric point between 11 and 13.

According to the invention, each variant maintains a bacteriostatic and/or bactericidal antibacterial activity, in particular an antibacterial activity against Gram negative bacteria and/or Gram positive bacteria.

According to the present invention, said bacteria are bacteria that are pathogenic for humans and/or animals.

In one embodiment, said Gram negative bacteria belong to one or more of the species Escherichia and/or Salmonella and said Gram positive bacteria belong to one or more of the species Listeria, Clostridium and/or Bacillus.

In a preferred embodiment, said bacteria belong to all the species listed above, and therefore said Gram negative bacteria belong to the species Escherichia and Salmonella and said gram positive bacteria belong to the species Listeria, Clostridium and Bacillus.

According to the present invention, said peptides exhibit bacteriostatic and/or bactericidal activity against several strains belonging to Escherichia coli and/or Salmonella thyphymurium strains, and said Gram positive may belong to Listeria innocua, Listeria monocytogenes, Bacillus thuringiensis, Clostridium sporogenes and/or Clostridium tyrobutyricum and/of Clostridium difficile strains.

According to the present invention, advantageously, one or more of said strains can be an antibiotic resistant strain, in particular, resistant to antibiotics selected from those commonly used in the breeding of poultry, cattle, pigs, sheep or even those commonly used in human and veterinary medical practice.

The present invention also relates to a bacteriostatic and/or bactericidal antibacterial composition comprising one or more peptide according to the present description or the claims and at least one suitable excipient and/or carrier.

In one embodiment the composition according to the present invention comprises the peptide having or one or more of the peptides having SEQ ID 1-4 or variants thereof as defined in the present description.

In a preferred embodiment, any composition according to the present invention comprises one or more of the peptides having SEQ ID NO 1 and SEQ ID NO 2.

In a particular embodiment, the only antibacterial agents in the composition of the invention are one or more of the peptides having SEQ ID 1-4 or variants thereof as defined in the present description.

As shown in the examples section, said peptides show effective bactericidal activity against various bacteria, both Gram positive and Gram negative.

The selection of carriers and/or excipients will depend on the intended use of the aforementioned composition.

In the event of an intended food use, said excipients and/or carriers will be represented by suitable edible excipients, commonly used in food practice, such as water, saline solutions, maltodextrins, starches.

In the event of an intended medical use, said compositions are pharmaceutical compositions and said excipients and/or carriers are of a pharmaceutically acceptable grade and can be selected from those commonly used in the pharmaceutical technique according to the type of desired composition.

The skilled person, when formulating the peptides of the invention will use excipients and/or carriers suitable for solid, granular, powder, liquid, semi-liquid formulations, emulsions, suspensions, solutions using common pharmacological practice.

The pharmaceutical compositions according to the invention can be for oral, parenteral or systemic use.

The invention hence relates to pharmaceutical compositions as defined above, for use as a medicament or, in the jurisdictions that allow it, methods of therapeutic treatments comprising the administration in pharmacologically effective doses of the composition of the invention to a subject, human or animal, in need thereof.

The aforesaid pharmaceutical composition may also comprise further pharmacologically active ingredients. According to a different embodiment, however, this composition will comprise only peptides according to the present invention or according to the claims as the sole pharmacologically active ingredients. In one embodiment, therefore, the pharmaceutical composition can comprise as the sole pharmacologically active ingredients the peptide having SEQ ID NO 1 and/or the peptide having SEQ ID NO 2 and/or the peptide having SEQ ID NO 3 and/or the peptide having SEQ ID NO 4 or variants thereof as defined in the present description.

In one embodiment, the pharmaceutical composition according to the description and according to the claims is a composition for the treatment, or to assist in the treatment, of bacterial infections due to Gram positive and/or Gram negative bacteria.

The invention also relates to the use of the composition as described above and as claimed or of one or more peptides as described and claimed, for the sanitization of environments or plants.

The peptides or the composition according to the invention can for example be diluted in suitable concentrations, sufficient to exert the antibacterial activity, in water or in hydroalcoholic solution to be, for example, nebulized in the environments of interest or to be nebulized or applied on surfaces of the same or on surfaces or components of plants that have to be sanitized.

The amount of peptides or composition will be such as to allow an effective bactericidal action. Given the non-toxicity of the peptides as defined and claimed in this description for humans and animals, amounts of peptides equal to their minimum bacterial growth inhibition concentrations as obtained by MIC assays can be used as dosages for treatments, but even higher concentrations.

A non-limiting example of environments that can be sanitized with the peptides or with the solution of the invention is represented by places for the breeding of animals, such as for example poultry, cattle, pigs, sheep, goats, rodents, dogs, etc., or hospitals, schools, kindergartens, medical or veterinary surgeries and the like.

In principle, given the non-toxicity of the peptides of the invention, the peptides as such or the composition that comprises them are suitable for use for the sanitation of environments used for the presence of humans or animals.

The invention also relates to a method for the antibacterial treatment of foods comprising mixing said foods or treating said foods with one or more peptides or with the composition according to the present description or according to the claims.

The antibacterial treatment according to the invention makes it possible to avoid treatments with substances such as antibiotics and/or of chemical synthesis, or even the heat treatment of foods, thereby allowing to obtain healthy foods without alternating their taste and organoleptic properties, while maintaining a suitable shelf-life of the food product.

A non-limiting example of foods that can be subjected to the antibacterial treatment according to the invention is represented by dairy products, such as cheeses, yoghurt, milk and the like, meat-based products, sausages, sauces and condiments, or fish-based products (fish, crustaceans, molluscs) and easily perishable food products that normally require treatments with synthetic compounds and/or heat treatments to slow down their decomposition and improving their conservation by extending their shelf-life.

Given their origin from edible plants commonly used in human and/or animal nutrition, said peptides or a suitable composition that includes them, as described here, can be placed in water for alimentary or breeding use in order to exert their bacteriostatic and/or bactericidal action. For example, water treatment can be carried out in tanks suitable for the collection or conservation of water, allowing the bacterial load of the water contained therein to be reduced.

Finally, the invention relates to food preparations comprising one or more peptide or the composition according to the present description or according to the claims. For food preparations processed from raw materials are ment, and are therefore excluded, for example, vegetables as such or plants elaborates from which said peptides are extracted.

A non-limiting example of food preparations can be represented for example by dairy products, including milk as such, cheese, yoghurt and the like, or even meat-based products such as sausages, ready-made sauces, and the like.

The uses described herein of the peptides or of the composition according to the present invention allow to obtain an effective control of the bacterial load of Gram positive and/or Gram negative bacteria, contributing, in fact, to human and animal health.

Below technical data obtained on the peptides tested and examples of embodiments of the invention are provided.

Peptides having SEQ ID NO 1-4 were recombinantly expressed in E. coli by standard techniques or artificially synthesized.

Finally, the invention also relates to a method for identifying peptides having bacteriostatic and/or bactericidal activity of natural origin, comprising the following steps

a. analysing the sequence of peptides of plant origin, preferably derived (extracted) from edible plants and selecting among these, peptides whose sequence has the following features:

a length from 18 to 27 amino acids wherein, from 30 to 40% of said amino acid sequence is composed of positively charged amino acids, from 9 to 14% of said amino acid sequence is composed of aromatic amino acids and said amino acid sequence is free of negatively charged amino acids;

b. calculatimg the net charge, the isoelectric point, the hydrophobicity and the Boman index of each peptide selected in point a. and further selecting peptides with a Boman index between 2 and 4, a net positive charge between 5.5 and 8, and a hydrophobicity between 0 and -1 and an isoelectric point between 11 and 13.

Preferably, according to the invention, the sequence of the peptides selected in point a. has, in particular, the following features: positively charged amino acids from 30 to 35%, and aromatic amino acids from 10 to 12.5%.

Preferably in point a. peptides having a length of 19 to 25 amino acids, or even peptides having a length of 20 to 24 amino acids, can be selected.

The method of the invention can further comprise one or more steps c. of measurement of the antibacterial activity, understood as bacteriostatic and/or bactericidal, of one or more peptides identified in point b.

The calculations in point b. can be carried out with standard methods commonly used by the person skilled in the art and can also be easily carried out using free programs available online.

Preferably, in any embodiment of the invention, the calculations can be carried out as described in Osorio et al 2015 and as reported in the examples section below.

The measurement of bacteriostatic and/or bactericidal activity can be carried out with conventional methods known to those skilled in the art, such as in the ways described above and described in the examples below. For example, said activity can be measured by calculating the MIC, which, as commonly known, allows to determine the minimum concentration of peptide capable of inhibiting the bacterial growth of the microorganism in question. The bacteriostatic and/or bactericidal activity can also be further analysed by means of challenge experiments as described in the examples section below, by means of the diffusion test on agar known in the art, by means of chromatography, known to those skilled in the art.

The invention also relates to peptides obtainable from the method described and claimed herein. An example of peptides obtainable from the method of the invention is represented by peptides having SEQ ID NO 1-4 or variants thereof as defined above.

The invention also relates to the use of peptides identified with the method described above or compositions that include them such as those described above for peptides having SEQ ID 1-4, as bacteriostatics and/or bactericidals.

### ESEMPI

1. Qualification Analysis of the Antimicrobial Action: MIC Analysis

The ability of each peptide having SEQ ID 1 to 4 to inhibit a pool of microbial species was tested in in vitro assays. These peptides respond to the selective parameters indicated in the present description and in the claims.

The peptides were individually tested against the following bacterial species: Escherichia coli, Listeria monocytogenes, Bacillus thuringiensis, Pseudomonas spp., Salmonella thyphymurium, C. botulinum/sporogenes, Clostridium tyrobutyricum, Clostridium difficile.

MIC assays were performed in 96-well plates in a total volume of 200 μl, with a peptide amount ranging from 1 μg/ml to 512 μg/ml. All assays were performed in triplicate. A negative control (growth medium only) and a positive control (medium plus microorganism) were included in each run. For the MIC assays on Gram+bacteria the ISO 10932: 2010 (E) protocol was followed, while for the Grams−the CLSI method was followed (M100-S19, Vol. 29, No. 3).

The absorbance values read after incubation at 620 nm, were then processed so to obtain inhibition percentages of the assayed peptides, which are shown in the table below.

Antimicrobial activity of the tested peptides

| | B. thuringiensis | | Listeria monocytogenes | | E. coli | |
|---|---|---|---|---|---|---|
| | MIC (μg/ml) | | MIC (μg/ml) | | MIC (μg/ml) | |
| Peptide | Range | MIC | Range | MIC | Range | MIC |
| Seq ID no1 | 1 a 512 | 512 | 1 a 512 | 256 | 1 a 512 | 128 |
| Seq ID no2 | 1 a 512 | 256 | 1 a 512 | 32 | 1 a 512 | 256 |
| Seq ID no3 | 1 a 512 | 128 | 1 a 512 | 512 | 1 a 512 | / |
| Seq ID no4 | 1 a 512 | / | 1 a 512 | 512 | 1 a 512 | 128 |

Antimicrobial activity of the tested peptides

| | Salmonella thyphymurium | | C. sporogenes | | C. tyrobutyricum | | C. difficile | |
|---|---|---|---|---|---|---|---|---|
| | MIC (μg/ml) | | MIC (μg/ml) | | MIC (μg/ml) | | MIC (μg/ml) | |
| Peptide | Range | MIC | Range | MIC | Range | MIC | Range | MIC |
| Seq ID no1 | 1 a 512 | 256 | 1 a 512 | 256 | 1 a 512 | 32 | 1 a 512 | >100 |
| Seq ID no2 | 1 a 512 | / | 1 a 512 | 4 | 1 a 512 | 1 | 1 a 512 | 1.5 |
| Seq ID no3 | 1 a 512 | / | 1 a 512 | 256 | 1 a 512 | 64 | 1 a 512 | / |
| Seq ID no4 | 1 a 512 | >512 | 1 a 512 | 512 | 1 a 512 | 16 | 1 a 512 | / |

MIC results of the tested peptides against the seven bacterial species tested. MIC indicates the minimum quantity in μg/ml of peptide capable of inhibiting the bacterial growth of the microorganism in question, tested in a range from 1 to 512 μg/ml.

The data obtained, shown in the table above, show that all the peptides tested exert a growth inhibition activity against one or more of the bacteria tested.

2. Challenge Test in Dairy Models

The peptides having SEQ ID NO 1 and SEQ ID NO 2 were tested in a food model. In particular, the peptides PV2 and SIORFs3663 were tested against B. thuringiensis.

The dairy model developed consists of a ricotta-based growth medium (called agar-ricotta). A culture medium consisting of 50% fresh ricotta available on the market, and the remaining 50% of 1% water agar was prepared. In order to evaluate bacterial growth, Trifenyltetrazolium chloride (TTC) was added at a final concentration of 0.005%. TTC is a biochemical indicator capable of highlighting cellular respiration as its colour changes to red. The tests were carried out in 96-well plates, in which about 200 pl of ricotta agar were dispensed into each well, and where different concentrations of the peptides were tested on a fixed concentration of bacterium (10⁶ CFU/ml), and a constant peptide concentration (512 μg/ml) on different microorganism dilutions (10⁴, 10³, 10², 10 CFU/ml). The peptides having SEQ ID 1 and SEQ ID 2 have been shown to inhibit levels equal to 10²-10³ CFU/ml of B. thuringiensis at a concentration of 512 μg/ml.

3. Challenge Test on Meat Models

The salami food model (called agar-salami) was made using an aliquot of 20 g of dough, consisting of 60% lean meat and 30% lard; to these are added 4 ml of sterile water (20% water on the minced meat) and the whole is added to 60 ml of 1.5% sterile water-agar (ratio 1:3 between mixture and agar). The mixture thus obtained is heated for a few seconds in the microwave and sterile filtered. This is followed by a subsequent heating in the microwave, followed by cooling until it reaches about 55° C. and the addition of glucose and TTC. The percentage of glucose is calculated on the final volume of mixture+agar, as well as that of TTC, and must be equal to 0.4%, while that of TTC must be 0.005%. The test is carried out in 25-well plates with a volume of salami agar, equal to 650 μl. In this food model the peptides were tested to verify the ability to inhibit the growth of Listeria monocytogenes. Different concentrations of the peptides (range 512-64 μg/ml) were applied to a fixed concentration of bacterium (10⁶ CFU/ml), and a constant concentration of the peptides, the same highlighted in MIC, was tested on different microorganism dilutions (10⁴, 10³, 10², 10 CFU/ml). The tests demonstrated the ability of both peptides to inhibit the growth of L. monocytogenes, confirming the MIC results also in the salami food model, i.e. the peptide having SEQ ID NO 1 at 256 μg/ml is able to inhibit 10² UFC/g of L. monocytogenes, while the peptide having SEQ ID NO 2 at 32 μg/ml is capable of exerting a bactericidal effect on L. monocytogenes concentrations equal to 10₂ CFU/g.

4. Peptide Cytotoxicity Analysis on Human 3T3 Fibroblasts

The potential cytotoxicity of peptides having SEQ ID NO 1 and SEQ ID NO 2 was evaluated on a 3T3 murine fibroblast cell line. Cells were cultured in DMEM medium (37° C., 5% CO2), containing 2 mM glutamine and 500 units/mL penicillin, 10 mg/mL streptomycin, supplemented with 10% Foetal Calf Serum inactivated until the moment of use. The cells were cultured in 96-well microplates (1×104 cells/well) for 24 hours in serum-free medium in order to synchronize cell division. Subsequently, the serum-free medium was removed and replaced with complete medium (100 μl) to which the peptides, previously dissolved in PBS, were added at increasing concentrations (the final concentrations in μg/ml are expressed in the figure below). To measure cell viability, evaluated at 24 and 48 hours, the CellTiter-Blue® assay (Promega, Madison, WI, USA) was applied according to the manufacturer's instructions. The fluorescence of the solution (560Ex/590Em) was determined with an automatic microplate reader (Glomax, Promega, WI, U.S.A.) after correct incubation with the reagent at 37° C.

Each experiment was conducted in triplicate. Data are shown as the mean±standard deviation. Statistical significance was measured with the ANOVA test. Dunnett's test (P values lower than 0.05) was applied to the entire dataset to compare the controls with the other groups.

The assay shows that at the MIC concentrations evaluated for the peptides tested, the peptides do not have a cytotoxic effect on human fibroblasts, even when placed directly in the culture medium. The peptide having SEQ ID NO 2 shows a certain degree of cytotoxicity starting from concentrations of 60μg/ml. MIC values higher than this concentration were found only against the microorganisms B. cereus and E. coli with MIC values equal to 256 μg/ml. Moreover, this peptide showed MIC values against the other microorganisms tested well below the concentrations found to be cytotoxic.

5. Calculation of Net Charge, Hydrophobocity, Boman Index and Isoelectric Point Values.

The calculations of the charge, hydrophobicity, Boman index, and isoelectric point values were calculated using the R Peptides package which is based on what reported by Osorio et al 2015.

Net charge was calculated using equation 1, below, which is a variation of the Henderson Hasselbalch equation proposed by Moore (1985) in which N are the number of amino acids, and the indices j represent, respectively, the amino acids (Aspartic Acid, Acid Glutamic, Cysteine and Tyrosine) and (Arginine, Lysine and Histidine).

The net charge of a protein can be calculated by specifying the pH value and one of nine available pKa scales (Bjellqvist, Dawson, EMBOSS, Lehninger, Murray, Rodwell, Sillero, Solomon or Stryer).

In this case the parameters used for each peptide were pH=7, and pK scale=“EMBOSS”.

$$charge = {\sum }_{i} {N}_{i} \frac{+ 1}{1 + \text{?}} + {\sum }_{j} {N}_{j} \frac{- 1}{1 + \text{?}} \qquad ( 1 )$$

$$\text{?} \text{indicates text missing or illegible when filed}$$

Boman Index

The potential protein interaction index has been proposed by Boman (2003) as a simple way to differentiate the mechanism of action of hormones (protein-protein) and antimicrobial peptides (protein-membrane) through this index. The index was calculated using equation 2 below, adding each amino acid solubility divided by the length of the sequence This function predicts the potential interaction of the peptide with another protein.

$${B}_{index} = \frac{{\sum }_{i = 1}^{N} {S}_{i}}{N} \qquad ( 2 )$$

Isoelectric Point

The isoelectric point (pI) is the pH at which the net charge of the protein equals 0. It is a variable that affects the solubility of peptides under certain pH conditions. When the pH of the solvent is equal to the pI of the protein, it tends to precipitate and lose its biological function.

The calculation of the isoelectric point of a peptide can be carried out using the Pi function by specifying one of the nine available pKa scales (Bjellqvist, Dawson, EMBOSS, Lehninger, Murray, Rodwell, Sillero, Solomon or Stryer). In the present case the EMBOSS scale was used

pI=1/2(pKₐ₁α-COOH+pKₐ₂α-NH₃⁺).

Hydrophobicity

Hydrophobicity is an important stabilizing force in protein folding; this force changes depending on the solvent in which the protein is located. It is considered the driving force of the peptide for the membrane core. The hydrophobicity index was calculated following Equation 3 below, adding the hydrophobicity of the individual amino acids and dividing this value by the length of the sequence.

$$H = \frac{{\sum }_{i = 1}^{N} {H}_{i}}{N} \qquad ( 3 )$$

1. An antibacterial peptide having a length from 18 to 27 amino acids, comprising: from 30 to 40% of positively charged amino acids, from 9 to 14% of aromatic amino acids and 0% of negatively charged amino acids.

2. The peptide according to claim 1 wherein said peptide is of vegetable origin, preferably derived from edible plants.

3. The peptide according to claim 1 wherein said positively charged amino acids are from 30 to 35% of said peptide and said aromatic amino acids are from 10 to 12.5% of said peptide.

4. The peptide according to any claim 1 wherein said peptide has a Boman index between 2 and 4, a net positive charge between 5.5 and 8, a hydrophobicity between 0 and −1 and an isoelectric point between 11 and 13.

5. The peptide according to claim 1 wherein said peptide exhibits antibacterial, bacteriostatic and/or bactericidal activity.

6. The peptide according to claim 5 wherein said antibacterial activity is towards Gram negative bacteria and/or towards Gram positive bacteria.

7. The peptide according to claim 6 wherein said peptide has SEQ ID NO 1, SEQ ID NO 2, SEQ ID NO 3 or SEQ ID NO 4, or variants thereof, wherein each of said variants is a peptide having a sequence identity of at least 80% compared to a peptide having SEQ ID NO 1, SEQ ID NO 2, SEQ ID NO 3 or SEQ ID NO 4, a length from 18 to 27 amino acids, and comprises: from 30 to 40% or from 30 to 35%, of positively charged amino acids, from 9 to 14% or 10 to 12.5%, of aromatic amino acids and 0% of negatively charged amino acids; has a Boman index between 2 and 4, a net positive charge included between 5.5 and 8, a hydrophobicity between 0 and −1 and an isoelectric point between 11 and 13.

8. The peptide according to claim 1 wherein said Gram negative bacteria belong to Escherichia and/or Salmonella species and said Gram positive bacteria belong to Listeria, Clostridium and/or Bacillus species.

9. The peptide according to claim 8 wherein said Gram negative bacteria belong to strains of Escherichia coli and/or Salmonella thyphymurium, and said Gram positive bacteria belong to strains of Listeria innocua, Listeria monocytogenenses, Bacillus thuringiensis, Clostridium sporogenses, Colostridium difficile and/or Colostridium tyrobutyricum.

10. The peptide according to claim 9 wherein said strains are resistant antibiotic strains.

11. A method for the identification of peptides having bacteriostatic and/or bactericidal activity of natural origin, including the following steps
a. analysing the sequence of peptides of plant origin, preferably derived from edible plants, and selecting among these, the peptides whose sequence has the following features:
a length from 18 to 27 amino acids in which, from 30 to 40% of said amino acid sequence consisting of positively charged amino acids, from 9 to 14% of said amino acid sequence consisting of aromatic amino acids and said amino acid sequence being free of negatively charged amino acids;
b. calculating the isoelectric point, hydrophobicity and Boman index of each peptide selected in point a. and further selecting peptides having a Boman index between 2 and 4, a net positive charge between 5.5 and 8, and a hydrophobicity between 0 and −1 and an isoelectric point between 11 and 13.

12. The method according to claim 11, wherein said positively charged amino acids in point a. are from 30 to 35%, and said aromatic amino acids in point a. are from 10 to 12.5% of said amino acid sequence.

13. The method according to claim 11 further comprising one or more steps c. measurement of the bacteriostatic and/or bactericidal activity of one or more peptides identified in point b.

14. Peptides having antibacterial activity obtainable by the method according to claim 11.

15. The peptides according to claim 14, wherein said peptides have SEQ ID NO 1, SEQ ID NO 2, SEQ ID NO 3 or SEQ ID NO 4 or are variants thereof, wherein each of said variants is a peptide having a sequence identity of at least 80% compared to a peptide having SEQ ID NO 1, SEQ ID NO 2, SEQ ID NO 3 or SEQ ID NO 4, a length from 18 to 27 amino acids, and comprises: from 30 to 40% or from 30 to 35%, of positively charged amino acids, from 9 to 14% or from 10 to 12.5%, of aromatic amino acids and 0% of negatively charged amino acids, has a Boman index between 2 and 4, a net charge positive between 5.5 and 8, a hydrophobicity between 0 and −1 and an isoelectric point between 11 and 13.

16. A antibacterial, bacteriostatic and/or bactericidal, composition comprising one or more peptides according to claim 1 and at least one excipient and/or carrier.

17. The composition according to claim 16 wherein said at least one excipient and/or carrier is pharmaceutically acceptable and wherein said composition is a pharmaceutical composition.

18. (canceled)

19. The pharmaceutical composition according to claim 17 further comprising one or more pharmacologically active ingredients.

20. A method of treating bacterial infections in a human or animal patient comprising administering a therapeutically effective amount of the pharmaceutical composition of claim 17 to said patient.

21. A method of sanitizing an environment or plant comprising contacting said environment or plant with an effective amount of the composition of claim 16.

22. The method according to claim 21 wherein said environments are animal farms, plants for food industries, hospital environments, environments for veterinary clinics.

23. A method for the antibacterial treatment of foods comprising mixing said foods or treating said foods with one or more peptides according to claim 1.

24. A method for sanitizing water comprising mixing said water or treating said water with one or more peptides according to claim 1.

25. Food preparations comprising one or more peptides according to claim 1.

26. The preparations according to claim 25 wherein said preparations are dairy or animal meat products or fish products.
